(19) United States
(12) Patent Application Publication
(10) Pub. No.: US 2008/0015141 A1
(43) Pub. Date: Jan. 17, 2008

(54) Use of a Novel Polymorphism in the Hsgk1 Gene in the Diagnosis of Hypertonia an Use of the Sgk Gene Family in the Diagnosis and Therapy of the Long Qt Syndrome

(76) Inventors: Florian Lang, Tubingen (DE); Andreas Busjahn, Panketal (DE)

Correspondence Address: FOLEY AND LARDNER LLP;SUITE 500, 3000 K STREET NW, WASHINGTON, DC, 20007, US

(21) Appl. No.: 10/544,576

(22) PCT Filed: Feb. 5, 2004
(86) PCT No.: PCT/EP04/01051

§ 371 Date: Mar. 8, 2006

(30) Foreign Application Priority Data
Feb. 7, 2003 (DE) 103 05 213.5

(51) Int. Cl. A61K 38/16; A61K 31/56; C12Q 1/68; G01N 33/00

(52) U.S. Cl. 514/8; 435/6; 436/501; 514/169

(57) ABSTRACT

The invention relates to the use of a single-stranded or double-stranded nucleic acid comprising a fragment of hsgk for diagnosing hypertension, with said fragment being at least 10 nucleotides/base pairs in length and with said fragment furthermore comprising a polymorphism with ensues from the presence or absence of an insertion of the nucleotide G at position 732/733 in intron 2 of the hsgk1 gene.

The invention furthermore relates to the use of the direct correlation between the overexpression or the functional molecular modification of human homologues of the sgk family and the length of the Q/T interval for diagnosing the long Q/T syndrome, and also to the use for the nucleic acid of a human homologue of the sgk gene family or of one of its fragments for diagnosing the long Q/T syndrome. In particular, polymorphisms of individual nucleotides (single nucleotide polymorphisms=SNP) in the human homologues of the sgk gene family can also, in the present case, be used for diagnosing a genetically determined predisposition for the long Q/T syndrome.

In a further aspect, the invention relates to the use of a functional activator or transcription factor which increases the expression of the genes of the sgk family for producing a pharmaceutical for the therapy and/or prophylaxis of the long Q/T syndrome.

The present invention relates to the use of a single-stranded or double-stranded nucleic acid containing an hsgk fragment for diagnosing hypertension, with said fragment being at least 10 nucleotides/base pairs in length and with said fragment furthermore comprising a polymorphism which ensues from the presence or absence of an insertion of the nucleotide G at position 732/733 in intron 2 of the hsgk1 gene.

The present invention furthermore relates to the use of the direct correlation between the overexpression or the functional molecular modification of human homologues of the sgk family and the length of the Q/T interval for diagnosing the long Q/T syndrome and also to the use of the nucleic acid of a human homologue of the sgk gene family or of one of its fragments for diagnosing the long Q/T syndrome. In particular, polymorphisms of individual nucleotides (single nucleotide polymorphisms=SNPs) in the human homologues of the sgk gene family can also, in the present case, be used for diagnosing a genetically determined predisposition for the long Q/T syndrome.

In a further aspect, the invention relates to the use of a functional activator or transcription factor which increases the expression of the genes of the sgk family for producing a pharmaceutical for the therapy and/or prophylaxis of the long Q/T syndrome.

Numerous extracellular signals lead to intracellular phosphorylation/dephosphorylation cascades for the purpose of ensuring rapid transfer of these signals from the plasma membrane and its receptors into the cytoplasm and the cell nucleus. The specificity of these reversible signal transfection cascades is made possible by a large number of individual proteins, in particular kinases, which transfer a phosphate group onto individual substrates.

The serum- and glucocorticoid-dependent kinase (sgk), which is a serine/threonine kinase whose expression is increased by serum and glucocorticoids, was initially cloned from rat mammary carcinoma cells (Webster et al., 1993). The human version of sgk, i.e. hsgk1, was cloned from liver cells (Waldegger et al., 1997). It was found that the expression of hsgk1 is influenced by regulating the cell volume. It has as yet not been possible to demonstrate such a dependence on the cell volume as far as expression of the rat sgk is concerned. It has furthermore been found that the rat kinase stimulates the epithelial Na⁺ channel (ENaC) (Chen et al., 1999; Naray-Pejes-Toth et al., 1999). The ENaC in turn plays a crucial role in renal Na⁺ excretion. An increase in the activity of the ENaC leads to an increase in the renal retention of sodium ions and, in this way, to the development of hypertension, as WO02/074987 A2 demonstrates.

Finally, two further members of the human sgk human family, i.e. hsgk2 and hsgk3, have been cloned (Kobayashi et al., 1999), both of which genes are, like hsgk1 as well, activated by insulin and IGF1 by way of the P13 kinase route. Electrophysiological experiments have shown that coexpression of hsgk2 and hsgk3 likewise results in a significant increase in the activity of the ENaC.

It is evident from DE 197 08 173 A1 that hsgk1 possesses substantial diagnostic potential in connection with many diseases in which changes in cell volume play a crucial pathophysiological role, such as hypernatremia, hyponatremia, diabetes mellitus, renal insufficiency, hypercatabolism, hepatic encephalopathy and microbial or viral infections.

WO 00/62781 reported that hsgk1 activates the endothelial Na⁺ channel, thereby increasing renal Na⁺ resorption. Since this increased renal Na⁺ resorption is accompanied by hypertension, it was presumed, in this case, that an increase in the expression of hsgk1 would lead to hypertension while a reduction in the expression of hsgk1 would ultimately lead to hypotension.

DE 100 421 37 also reported a similar connection between the overexpression or hyperactivity of the human homologues hsgk2 and hsgk3 and the hyperactivation of the ENaC, the increase in renal Na⁺ resorption resulting therefrom and the hypertension which develops from this. Furthermore, this document already discussed the diagnostic potential of the kinases hsgk2 and hsgk3 with regard to essential hypertension.

WO02/074987 A2 discloses the connection between the occurrence of two different polymorphisms (single nucleotide polymorphism (SNP)) of individual nucleotides in the hsgk1 gene and a genetically determined predisposition for hypertension. In this case, the polymorphisms are a polymorphism in intron 6 (T→C) and a polymorphism in exon 8 (C→T) in the hsgk1 gene. In particular, it is evident from Table 5 in WO02/074987 A2 that there is a strong correlation disequilibrium between the two SNPs which had been analyzed: most CC carriers of the SNP in exon 8 are also intron 6 TT carriers (namely 64%) whereas only a few exon 8 TT carriers are also at the same time intron 6 CC carriers (only 2%). These correlations which had been observed between the occurrence of the polymorphisms in intron 6 and exon 8 substantiate the necessity of analyzing the genotype for the two polymorphisms (intron 6 and exon 8) in order to demonstrate a predisposition for hypertension, with this leading to a high degree of technical input and time consumption.

The object of the present invention is therefore to provide a further polymorphism in the hsgk1 gene, the occurrence of which in one or the other version may correlate even better than the two known polymorphisms in exon 8 and intron 6 with the phenotypic occurrence of hypertension in the patient. In particular, the provision of a single SNP which correlates with the predisposition for hypertension and whose presence in one or the other version even has consequences for a functional molecular modification of the hsgk1 protein would be very advantageous.

This object was achieved by providing a novel polymorphism in the hsgk1 gene, which polymorphism comprises the insertion of the nucleotide G at position 732/733. It has been found that individuals which possess such an insertion of the nucleotide G at position 732/733 (InsG/InsG) occur more frequently and have a lower predisposition for developing hypertension. On the other hand, individuals which do not possess such an insertion at position 732/733 (WT/WT) occur more rarely and have a markedly higher predisposition for developing hypertension. According to the results obtained thus far, this correlation between the genotype, in regard to the polymorphism at position 732/733 in intron 2, and the predisposition to the development of hypertension appears to have a markedly higher significance than the corresponding correlations with regard to the polymorphisms in exon 8 and intron 6 (see Table 1).

Furthermore, it is to be assumed, on the basis of the prediction obtained using known prediction programs, that the expression of a specific splice variant of the hsgk1 gene depends on the presence or absence of the G insertion at position 732/733 in intron 2 of the hsgk1 gene. The expression of such a specific splice variant of the hsgk1 gene could result in a functional molecular modification of the hsgk1 protein, which leads to the hsgk1 activity being modified, in particular to the hsgk1 activity being increased. The physiological consequences of this molecular modification of the hsgk1 protein, in particular an increase in the activity of the hsgk1, could then ultimately result in the development of the symptoms of hypertension.

In a first aspect, the invention relates to the use of an isolated single-stranded or double-stranded nucleic acid which comprises a fragment of the nucleic acid sequence as depicted in SEQ ID No. 1 or as depicted in SEQ ID No. 2 for diagnosing hypertension, with said fragment being at least 10 nucleotides/base pairs, preferably at least 15 nucleotides/base pairs, in particular at least 20 nucleotides/base pairs, in length and with said fragment comprising the polymorphism in intron 2 of the hsgk1 gene either with or without the insertion of the nucleotide G at position 732/733.

SEQ ID No. 1 describes the genomic DNA sequence of hsgk1 without the insertion of nucleotide G (or GTP) at position 732/733 in intron 2 of the hsgk1 gene, i.e. what is termed the “wild-type (WT)” sequence, and SEQ ID No. 2 describes the genomic DNA sequence of hsgk1 with the insertion of nucleotide G (or GTP) at position 732/733 in intron 2 of the hsgk1 gene, i.e. what is termed the “insertion G (InsG)” sequence.

In a second aspect, the present invention relates to a kit for diagnosing hypertension, which kit comprises at least one isolated single-stranded or double-stranded nucleic acid which comprises a fragment of the sequence as depicted in SEQ ID No. 1 or 2. In this connection, said fragment from SEQ ID No. 1 or 2 is at least 10 nucleotides/base pairs, preferably at least 15 nucleotides/base pairs, in particular at least 20 nucleotides/base pairs, in length. Furthermore, said fragment from SEQ ID No. 1 or 2 should comprise the polymorphism in intron 2 of the hsgk1 gene either with or without the insertion of the nucleotide G at position 732/733.

Alternatively, the kit for diagnosing hypertension can, in addition to, or instead of, the abovementioned single-stranded or double-stranded nucleic acid, also comprise at least one antibody which is directed against such a region of the hsgk protein whose presence in the hsgk1 protein depends on the presence of the insertion of the nucleotide G at position 732/733 in intron 2 of the corresponding encoding hsgk gene. If, for example, the presence of the G insertion at position 732/733 in the hsgk1 gene were to induce the splicing-out of an exon, an antibody which was directed against precisely this spliced-out protein region could be used for detecting the polymorphism version of the individual. Such an antibody could be used, therefore, to diagnose a predisposition for developing hypertension.

In a third aspect, the invention relates to a method for diagnosing hypertension, which method comprises the following procedural steps:
a) withdrawing a body sample from an individual,
b) where appropriate, isolating and/or amplifying genomic DNA, cDNA or mRNA from the body sample according to a),
c) quantifying the alleles which possess an insertion of the nucleotide G at position 732/733 in intron 2 of the hsgk1 gene.

In step a), a body sample is withdrawn from a test individual which is preferably a mammal, in particular a human. In this diagnostic method according to the invention, the body samples from a patient which are preferably used are blood samples or saliva samples which comprise cellular material and which can be obtained from the patient with relatively little effort. However, other body samples which likewise comprise cells, such as tissue or cell samples or the like, can also be used.

In step b), standard methods (Sambrook J. and Russell D. W. (2001) Cold Spring Harbor, N.Y., CSHL Press) are used to prepare, where appropriate, and/or amplify, where appropriate, either genomic DNA or cDNA or else mRNA from the body sample from a). In this connection, it is possible to use any suitable methods which are familiar to the skilled person, It is also possible, where appropriate, to dispense with this DNA isolation step or DNA amplification step, in particular when use is made, in step c) of detection methods which themselves involve a PCR amplification step.

In step c), the number of alleles which possess an insertion of nucleotide G at position 732/733 in intron 2 of the hsgk1 gene is finally quantified. In this connection, those individuals which possess two WT alleles ought to have a predisposition for developing hypertension. The quantification/identification of the alleles with regard to the polymorphism at position 732/733 in intron 2 of the hsgk1 gene can be effected by using a variety of methods which are known to the skilled person. Some preferred methods are explained in more detail below. However, the quantification of the number of alleles which possess an insertion of nucleotide G at position 732/733 in intron 2 of the hsgk1 gene is not restricted to the following preferred methods which are described below. The genotype (or the number of alleles) can preferably be identified, with regard to the polymorphism at position 732/733, by directly sequencing the DNA, preferably the genomic DNA, from the body sample at said position 732/733 in intron 2 of the hsgk1 gene. To do this, it is necessary to use known sequencing methods to make available, as sequencing primers, short oligonucleotides which possess sequences from the immediate vicinity of position 732/733 of the hsgk1 gene.

Any known methods which are based on hybridizing the genomic DNA from the body sample with specific hybridization probes constitute further methods, which are likewise preferred, for identifying the genotype (or for quantifying the number of alleles) with regard to the polymorphism at position 732/733.

Southern blotting is an example of such a hybridization method. If, for example, the presence of the G insertion at position 732/733 in intron 2 of the hsgk1 gene were to destroy or else form a cleavage site for a restriction endonuclease, it would be possible to use specific hybridization probes to detect nucleic acid fragments having lengths which differ from the corresponding fragment lengths in the WT allele. In this way, it would be possible to detect a genotype which was specific with regard to the polymorphism in question at position 732/733.

If, as a result of the presence or absence of the G insertion at position 732/733, an alternative splice variant which lacks a particular exon were to be expressed, it would also be possible to detect the genotype, with regard to the polymorphism at position 732/733 in question, using a specific hybridization probe from the exon which was missing in the splice variant.

Another example of a hybridization method is that of hybridizing the genomic DNA from the body sample with a labeled, single-stranded oligonucleotide which is preferably 15-25 nucleotides in length and which either does or does not possess a G insertion at position 732/733. Under very specific hybridization conditions, which can be tested experimentally for each individual oligonucleotide using known methods, it is possible to distinguish a completely hybridizing oligonucleotide from an oligonucleotide having one single base mismatch.

Other preferred methods for identifying the genotype (or for quantifying the number of alleles) with regard to the polymorphism at position 732/733 are, in particular, the PCR oligonucleotide elongation assay or the ligation assay.

In the case of the PCR oligonucleotide elongation assay, it would be possible, for example, to provide an oligonucleotide which possesses the sequence of a fragment from SEQ ID No. 2 and, at its 3′ end, the G at the polymorphism position 732/733. When this oligonucleotide was hybridized with a sample fragment of the WT allele (without G insertion), it would not be possible to extend, and ultimately amplify, this fragment in a subsequent PCR reaction because of the mismatch at the 3′ end. On the other hand, when this oligonucleotide was hybridized with an InsG allele, it would be possible, because of the perfect base pairing at the 3′ end of the oligonucleotide, to achieve elongation and ultimately to obtain a PCR amplification product

A ligation assay is ultimately based on the same principle as the PCR oligonucleotide elongation assay: only those double-stranded nucleic acid fragments which possess an exact base pairing at their end can be ligated to another double-stranded nucleic acid fragment. The appearance of a specific ligation product can therefore be made dependent on the presence or absence of the G insertion at position 732/733 in intron 2 of the hsgk1 gene.

In addition to the correlation of the polymorphism according to the invention with the predisposition for hypertension, a second correlation of the polymorphism according to the invention was surprisingly found with the length of what is termed the Q/T interval. Markedly shorter Q/T intervals are seen in individuals which possess a WT/WT genotype with regard to position 732/733 in intron 2 of the hsgk1 gene than in individuals which possess an InsG/InsG genotype. Heterozygous (WT/InsG) individuals possess intermediate Q/T intervals (see Table 3). A significantly extended Q/T interval leads to the development of what is termed the long Q/T syndrome, which can manifest itself in cardiac rhythm disturbances, by way of ventricular fibrillation through to sudden cardiac death. Individuals possessing the InsG/InsG genotype ought therefore to have a predisposition for developing the long Q/T syndrome.

Because of the direct correlation, which has been demonstrated, between the length of the Q/T interval and the genetic makeup of the hsgk1 gene, in particular between the length of the Q/T interval and the polymorphism at position 732/733 in intron 2 of the hsgk1 gene, it is to be assumed that nucleic acids of another human homologue of the sgk family are likewise suitable for diagnosing the long QT syndrome.

The Q, R and S waves which can be detected using an ECG measuring instrument constitute experimental values for assessing depolarization. The Q/T interval is defined as the time which is to be detected, using an ECG measuring instrument, from the beginning of the propagation of the T wave (the appearance of the Q deflection) to the end of depolarization which is characterized by the end of the T wave. The Q/T interval therefore constitutes the time which elapses between the beginning of a new state of excitation of the heart and the return to the resting state. A markedly extended Q/T interval accordingly leads to cardiac rhythm disturbances and, ultimately, to the long Q/T syndrome which has already been mentioned.

The invention also relates, therefore, to the use of the direct correlation between the overexpression or functional molecular modification of human homologues of the sgk family, in particular of the hsgk1 gene, and the length of the Q/T interval for diagnosing the long QT syndrome.

A human homologue of the sgk family, which homologue encompasses, in the above sense, a functional molecular modification, is understood, in this connection, as being a homologue of the sgk family which is mutated in such a manner that the properties, in particular the catalytic properties or the substrate specificity, of the corresponding protein are altered.

The direct correlation, according to the invention, between the Q/T interval and the genetic makeup of the human homologues of the sgk family implies that it would be possible for individual mutations in the genes hsgk1, hsgk2 or hsgk3 to occur in individual patients, with these mutations modifying the level of expression or functional properties of the kinases hsgk1, bsgk2 or hspk3 and, in this way, leading to a genetically occasioned prolongation of the Q/T interval and, ultimately, to a predisposition for the development of the long Q/T syndrome. Such mutations could, for example, occur in the regulatory gene regions or else in intron sequences of the sgk gene locus. On the other hand, the individual differences in the genetic makeup of the sgk locus could also affect the coding region of the gene. Mutations in the coding region could then, where appropriate, lead to a functional change in the corresponding kinase, as, for example, to the catalytic properties of the kinase being modified, with these modified properties also ultimately influencing the Q/T interval. Accordingly, both the above-described types of mutation could bring about a prolongation of the Q/T interval and thereby, ultimately, predisposition for development of the long Q/T syndrome.

The above-described mutations in the human homologues of the sgk family, which bring about the predisposition for development of the long Q/T syndrome in the patient, are as a rule what are termed single nucleotide polymorphisms (SNPs) either in the exon region or in the intron region of these homologues. In their less frequently occurring version, termed the mutated version in that which follows, SNPs in the exon region of the hsgk genes can, where appropriate, give rise to amino acid substitutions in the corresponding hsgk protein and consequently lead to the kinase being functionally modified. In their mutated version, SNPs in the intron region or in regulatory sequences of the hsgk genes can, where appropriate, lead to a change in the level of expression of the corresponding kinase. However, SNPs in the intron region could also lead to a functional modification of the kinase if they affect the alternative splicing of the immature mRNA.

The invention also relates to the use of a single-stranded or double-stranded nucleic acid which comprises the sequence of a human homologue of the sgk family or one of its fragments, in particular the hsgk1 gene itself or one of its fragments, for diagnosing a predisposition for developing the long Q/T syndrome. In this connection, the single-stranded or double-stranded nucleic acid preferably has a length of at least 10 nucleotides/base pairs.

In addition to the abovementioned single-stranded or double-stranded nucleic acids, certain antibodies which are directed against substrates of the human homologues of the sgk family, in particular against substrates of hsgk1, are also suitable for diagnosing a predisposition for developing the long Q/T syndrome and hypertension. These diagnostic antibodies are preferably directed against an epitope of the human homologues of the sgk family, in particular of hsgk1, which contains the phosphorylation site of the substrate either in phosphorylated form or in unphosphorylated form.

In a preferred embodiment, the ubiquitin protein ligase Nedd4-2 (Acc No. BAA23711) is used as the substrate of the human homologue of the sgk family, This ubiquitin protein ligase is a protein which is specifically phosphorylated by the human homologues of the sgk family [Debonneville et al., Phosphorylation of Nedd4-2 by Sgk 1 regulates epithelial Na(+) channel cell surface expression. EMBO J., 2001; 20: 7052-7059; Snyder et al., Serum and glucocorticoid-regulated kinase modulates Nedd4-2-mediated inhibition of the epithelial Na(+) channel. J. Biol. Chem. 2002, 277; 5-8]. Phosphorylation sites for hsgk1 possess the consensus sequence (R X R X X S/T) where R is arginine, S is serine, T is threonine and X is any arbitrary amino acid. In Nedd4-2 (Acc No. BAA23711) there are two potential phosphorylation sites for hsgk1 which the abovementioned consensus sequence fits: the serine at amino acid position 382 and the serine at amino acid position 468.

The abovementioned antibodies for diagnosing a predisposition for developing the long Q/T syndrome are therefore preferably directed against the substrate Nedd4-2 and, particularly preferably, against a region of the Nedd4-2 protein which possesses the sequence of the potential phosphorylation site for hsgk1, i.e. the consensus sequence (R X R X X S/T). In particular, these antibodies are directed against Nedd4-2 protein regions which encompass at least one of the two potential phosphorylation sites serine at amino acid position 382 and/or serine at amino acid position 468.

The invention furthermore relates to a kit for diagnosing the long QT syndrome or other diseases which manifest themselves in a prolongation of the Q/T interval. This kit for diagnosing the long QT syndrome preferably comprises antibodies which are directed against the human homologues of the sgk protein family or, in particular, nucleic acids which are able to hybridize, under stringent conditions, with the human homologues of the sgk gene family. The kit can also jointly comprise antibodies which are directed against the human homologues of the sgk protein family and nucleic acids which hybridize, under stringent conditions, with the human homologues of the sgk gene family. Particularly preferably, the kit according to the invention for diagnosing the long Q/T syndrome can also comprise antibodies which are directed against the hsgk1 protein or nucleic acids which are able to hybridize, under stringent conditions, with the hsgk1 gene.

In this connection, a hybridization under stringent conditions is understood as meaning a hybridization under those hybridization conditions, with regard to hybridization temperature and formamide content in the hybridization solution, which have been described in relevant specialist literature (Sambrook J. and Russell D. W. (2001) Cold Spring Harbor, N.Y., CSHL Press).

In particular, the diagnostic kit can comprise, as hybridization probes, single-stranded or double-stranded nucleic acids which possess a sequence as depicted in SEQ ID No. 1 or 2, which are at least 10 nucleotides/base pairs in length and which encompass the polymorphism at position 732/733 in intron 2 of the hsgk1 gene either with or without the insertion of the nucleotide G.

The diagnostic kit according to the invention provides, in particular, antibodies which are specifically directed against those regions of the hsgk1 protein whose presence in the hsgk1 protein depends on the presence of the G insertion at position 732/733 in intron 2 of the hsgk1 gene. In particular, those regions which, due to the presence or absence of this G insertion in the immature mRNA, are spliced out alternatively, and are therefore not present in the mature mRNA and in the protein arising from it, are suitable for use as immunogenic epitopes against which diagnostic antibodies can be directed. Correspondingly, precisely those nucleic acid regions of the hsgk1 gene which are able to hybridize with such a gene region which is spliced out in dependence on the 0 insertion at position 732/733 are also suitable for use as diagnostic hybridization probes.

The kit for diagnosing the long Q/T syndrome can also preferably comprise, as specific hybridization probes, nucleic acid fragments which encompass the known SNPs in the hsgk1 gene, in particular the SNP in exon 8 (C2617T, D240D), the SNP in intron 6 CM71C) and/or the SNP in intron 2 at position 732/733 (insertion of G).

The correlation, which has been demonstrated within the context of the invention, between the genetic makeup of the genes of the hsgk1 gene family and the length of the Q/T interval also makes it possible to use functional activators, or positive transcription regulators, of the sgk family therapeutically for treating the long Q/T syndrome and similar diseases which are likewise accompanied by a prolonged Q/T interval. In this connection, a “functional activator” is understood as being a substance which activates the physiological function of the corresponding kinase of the sgk family. A “positive transcription regulator” is understood as being a substance which activates the expression of the corresponding kinase of the sgk family.

The invention consequently also relates to the use of a functional activator, or a positive transcription regulator, of a human homologue of the sgk family, in particular of hsgk1, for lowering the Q/T interval and, in particular, for therapy and/or prophylaxis of the long QT syndrome. Known functional activators and/or positive transcription regulators of the human homologues of the sgk family, in particular of hsgk1, are glucocorticoids, mineralocorticoids, aldosterone, gonadotropins and a number of cytokines, in particular TGF-β.

The invention therefore furthermore relates to the use of substrates selected from the group of substances comprising glucocorticoids, mineralocorticoids, aldo-sterone, gonadotropins and cytokines, in particular TGF-β, for producing a pharmaceutical for the therapy and/or prophylaxis of the long QT syndrome. The invention also relates to a pharmaceutical which comprises a substance selected from the abovementioned group of substances for the therapy and/or prophylaxis of the long Q/T syndrome.

The present invention is explained in detail by means of the following examples.

### EXAMPLE 1

A correlation study, in which the genotype of the hsgk1 gene in different patients (twins) was compared with the systolic and diastolic blood pressure values which were measured in these patients, and then statically evaluated, was carried out within the context of the present invention.

75 dizygotic pairs of twins were used for the correlation analysis (Busjahn et al., J. Hypertens 1996, 14: 1195-1199; Busjahn et al., Hypertension 1997, 29: 165-170). The experimental subjects all belonged to the German-Caucasian race and originated from different parts of Germany. Blood was taken from the pairs of twins, and from their parents, for the purpose of verifying the dizygotism and for further molecular genetic analyses. Each of the experimental subjects underwent a prior medical examination. None of the experimental subjects was known to be suffering from any chronic medically recognized disease. After 5 min, the blood pressure of the test subject, whose was in the sitting position, was measured by a trained physician using a standardized mercury sphygmomanometer (2 measurements at a time interval of 1 min). The mean of the two measurements was used as the blood pressure value.

The advantage of using dizygotic twins for correlation studies is that they are of the same age and that the external influences on their phenotypes are to be judged as being minimal (Martin et al., Nat Genet 1997, 17: 387-392).

The importance of studies on twins for the elucidation of complex genetic diseases was recently described by Martin et al., 1997.

The dizygotism of the pairs of twins was confirmed by using the polymerase chain reaction (PCR) to amplify five microsatellite markers. In this analysis of microsatellite markers, deoxyribonucleic acid (DNA) fragments are amplified by PCR using specific oligonucleotides which contain regions which are highly variable in different human individuals. The high degree of variability in these regions of the genome can be detected by means of slight differences in sizes of the amplified fragments, resulting, when there is diversity at the corresponding gene locus, in double bands, i.e. what are termed microsatellite bands, being formed after the PCR products have been subjected to gel-electrophoretic fractionation (Becket et al., J. Reproductive Med 1997, 42: 260-266).

For the purpose of carrying out a molecular genetic analysis of the target gene, in the present case the hsgk1 gene, three further microsatellite marker regions (d6s472, d6s1038, d6s270) in the immediate vicinity of the hsgk1 locus were amplified by PCR and then compared with the corresponding samples from the other twin and the parents. In this way, it was possible to decide whether the twins had inherited alleles, from their parents, which were identical or different with regard to the allele under investigation. The correlation analysis was carried out using the structural equation modelling (SEM) model (Eaves et al., Behav Genet 1996, 26: 519-525; Neale, 1997: Mx: Statistical modeling, Box 126 MCV, Richmond, Va. 23298; Department of Psychiatry. 4th edition). This model is based on variance-covariances matrices of the test pairs which are characterized by the probability that they possess either no, one or two identical alleles. The variance with regard to the phenotype was divided into a variance which is based on the genetic background of all the genes (A), a variance which is based on the genetic background of the target gene (Q), in this case the hsgk1 gene, and the variance due to external influences (E).

VAR=A²+Q²+E²

The covariance of a test pair was defined for the three possible allele combinations IBD₀, IBD₁ and IBD₂ (IBD=identical by descent; 0, 1 or 2 identical alleles) as follows:

COV(IBD₀)=0.5A²

COV(IBD₁)=0.5A²+0.5Q²

COV(IBD₂)=0.5A²+Q²

In order to evaluate the correlation between the genetic makeup of the hsgk1 locus and the blood pressure of the test subject, the differences between models which do and, respectively, do not take into account the genetic variance with regard to the target gene hsgk1 were calculated as χ² statistic. For each pair and each gene locus, the allele ratios were calculated by means of the so-called multipoint model (MAPMAKER/SIBS; Kruglyak et al., Am J Hum Genet 1995, 57: 439-454) based on the parental genotypes.

The greater informative value of the analytical method which is based on a variance-covariance evaluation, as compared with the above-described χ² statistic (S.A.G.E. Statistical Analysis for Genetic Epidemiology, Release 2.2. Computer program package, Department of Epidemiology and Biostatistics, Case Western Reserve University, Cleveland, Ohio, USA, 1996) was recently confirmed in a simulation study (Fulker et al., Behav Gen 1996, 26: 527-532). An error probability of p<0.01 was accepted in order to ensure a significant correlation with regard to the criteria of Lander and Kruglyak (Lander et al., Nat Genet 1995, 11: 241-246).

Table 1 shows the results of this correlation study,

TABLE 1

| Phenotype | max χ² | p |
|---|---|---|
| Systolic blood pressure value (lying) | 4.44 | 0.04 |
| Diastolic blood pressure value (lying) | 14.36 | 0.0002 |
| Systolic blood pressure value (sitting) | 5.55 | 0.019 |
| Diastolic blood pressure value (sitting) | 4.92 | 0.027 |
| Systolic blood pressure value (standing) | 1.91 | 0.17 |
| Diastolic blood pressure value (standing) | 4.83 | 0.028 |

As can be seen from Table 1, the low values for the ascertained error probabilities p, which do not exceed, or only slightly exceed, the accepted error probability of p<0.01, prove that there is a direct correlation between the genetic variance with regard to the hsgk1 gene locus and the phenotypically ascertained variance of the measured blood pressure.

### EXAMPLE 2

The genomic organization of the hsgk1 gene has already been described (Waldegger et al., Genomics, 51, 299 [1998]), http://www.ensembl.org/Homo_sapiens/geneview?gene=ENSG00000118515).

In order to identify SNPs whose occurrences are relevant for a predisposition for developing hypertension, the SNPs in the hsgk1 gene which were published in databases were first of all investigated in order to determine whether they are genuine SNPs, and not simple sequencing errors, and whether the SNPs are sufficiently polymorphic in order to form the basis for a diagnostic detection of a predisposition for hypertension. The SNP rs 1057293 in exon 8, which concerns the replacement of a C with a T (http://www.ensembl.org/Homo_sapiens/snpview?snp=1057293; http://www.ncbi.nln.nih.gov/SNP/snp_ref.cgi?type=rs&rs=1057293) and a second SNP, which is located in the hsgk1 gene, at a distance of precisely 551 bp from the first SNP, in the donor splice site of intron 6 to exon 7 and concerns the replacement of a T with a C, had already been located in this way.

### EXAMPLE 3

Blood samples were taken from a random sample of the 75 pairs of twins. After the genomic DNA of the hsgk1 gene had been amplified from the blood samples by means of PCR, the exons and introns (but not the promoter region) of the hsgk1 gene were sequenced directly and completely using suitable sequencing priers. When the sequences of the hsgk1 genes which originated from different test subjects were compared, a further polymorphism in intron 2, consisting of the insertion of an additional nucleotide G in position 732/733, was noted. Furthermore, the presence or absence of this G insertion at position 732/733 in the hsgk1 genes of the individual test subjects exhibited a significant correlation with the blood pressure which was measured in the individual test subjects: on average, InsG/InsG genotypes exhibited significantly lower systolic and diastolic blood pressure values that did the less frequent WT/WT genotypes as well as the heterozygous WT/InsG genotypes (see Table 3). By contrast, other polymorphisms in the hsgk1 gene exhibited a correlation with the measured blood pressure which was less significant (e.g. intron 6 (C2071T) and exon 8 (T2617C, D240D)) or else no correlation with the measured blood pressure (e.g. intron 3 position Ins 13+xT, T1300-1312 and intron 4 (C1451T) and intron 7 position 2544delA), as Table 2 shows.

The ECG values, which were likewise measured on the test subjects, also showed that there was a marked correlation of the Q/T intervals, which were determined for the individual test subjects, with the genotype of the test subjects with regard to the polymorphism in intron 2 at position 732/733 of the hsgk1 gene: in this connection, test subjects possessing the less frequent WT/WT genotype exhibited markedly shorter Q/T intervals than heterozygous WT/InsG test subjects, while these latter in turn exhibited significantly shorter Q/T intervals than did test subjects possessing the more frequent InsG/InsG genotype (see Table 3). Longer Q/T intervals increase the danger of contracting cardiac rhythm disturbances, such as, in particular, the long Q/T syndrome. Consequently, inverse correlations are found between the genotype of the polymorphism in intron 2 at position 732/733 of the hsgk1 gene and a predisposition for the long Q/T syndrome, on the one hand, and a predisposition for hypertension, on the other hand. These correlations can in each case be used for the diagnosis, therapy and prophylaxes of hypertension and the long Q/T syndrome.

TABLE 2

| | intron 2 | intron 3 | | | intron 7 | |
|---|---|---|---|---|---|---|
| SNP/ | position | position | | | position | exon 8 |
| DNA | insG | ins13 + xT | intron 4 | intron 6 | delA | T2617C, |
| No. | 732\hat{ }733 | T1300\hat{ }1312 | C1451T | C2071T | 2544delA | D240D |
| 1899 | wt/wt | ins13 + xT | C/C | C/T | wt/wt | T/C |
| 2022 | wt/wt | ins13 + xT | C/C | C/C | wt/wt | C/C |
| 2094 | insG/wt | ins13 + xT | C/C | C/C | wt/wt | T/T |
| 1902 | insG/wt | ins13 + xT | C/C | T/T | wt/wt | C/C |
| 2041 | wt/wt | ins13 + xT | C/C | C/C | wt/wt | C/C |
| 2108 | insG/wt | ins13 + xT | C/C | C/T | wt/wt | T/C |
| 1921 | insG/wt | ins13 + xT | C/C | C/T | delA/wt | C/C |
| 2048 | insG/wt | ins13 + xT | C/C | T/T | wt/wt | C/C |
| 2115 | wt/wt | ins13 + xT | C/C | C/T | wt/wt | T/C |
| 1934 | insG/wt | ins13 + xT | C/C | C/T | wt/wt | T/C |
| 2049 | insG/wt | ins13 + xT | C/C | C/T | wt/wt | C/C |
| 2133 | insG/ | ins13 + xT | C/C | T/T | wt/wt | C/C |
| | insG | | | | | |
| 1944 | wt/wt | ins13 + xT | C/C | C/T | wt/wt | C/C |
| 2072 | insG/ | ins13 + xT | C/C | T/T | wt/wt | C/C |
| | insG | | | | | |
| 2159 | insG/wt | ins13 + xT | C/C | T/T | wt/wt | C/C |
| 1983 | wt/wt | ins13 + xT | C/C | C/C | wt/wt | T/C |
| 2076 | insG/wt | ins13 + xT | C/C | C/T | wt/wt | T/C |
| 2166 | wt/wt | ins13 + xT | C/C | C/C | wt/wt | T/C |
| 2011 | wt/wt | ins13 + xT | C/C | C/C | wt/wt | T/C |
| 2084 | insG/wt | ins13 + xT | C/C | C/T | wt/wt | C/C |
| 2278 | wt/wt | ins13 + xT | C/C | C/C | wt/wt | T/T |
| 2020 | insG/ | ins13 + xT | C/C | T/T | wt/wt | C/C |
| | insG | | | | | |
| 2085 | wt/wt | ins13 + xT | C/C | C/T | wt/wt | T/C |
| 2338 | insG/ | ins13 + xT | C/T | T/T | wt/wt | C/C |
| | insG | | | | | |

TABLE 3

| Measured | | | | |
|---|---|---|---|---|
| quantity/genotype | wt/wt | wt/ins | ins/ins | Significance |
| (Mean ± standard | n = 7 | n = 14 | n = 7 | |
| deviation) | | | | |
| Systolic blood pressure | 123 ± 17 | 116 ± 10 | 117 ± 15 | <0.05 |
| Diastolic blood | 73 ± 14 | 70 ± 9 | 72 ± 9 | n.s. |
| pressure | | | | |
| Q/T interval | 403 ± 13 | 411 ± 17 | 428 ± 10 | <0.05 |

1.-20. (canceled)

21. A method of using an isolated single-stranded or double-stranded nucleic acid comprising a fragment of the nucleic acid sequence depicted in SEQ ID No. 1 or depicted in SEQ ID No. 2 for diagnosing hypertension in vitro, wherein said fragment:
(a) is at least 10 nucleotides/base pairs in length; and
(b) comprises the polymorphism in intron 2 of the hsgk1 gene either with or without the insertion of the nucleotide G at position 732/733.

22. A kit for quantitatively diagnosing hypertension, comprising at least one isolated single-stranded or double-stranded nucleic acid as defined in claim 21.

23. A kit for quantitatively diagnosing hypertension, comprising at least one antibody directed against a region of the hsgk protein, characterized in that the presence of said region in the hsgk1 protein depends on the presence of an insertion of the nucleotide G at position 732/733 in intron 2 of the encoding hsgk gene.

24. A method for diagnosing hypertension in vitro, comprising the following procedural steps:
(a) withdrawing a biological sample; and
(b) quantifying in the biological sample the alleles which possess an insertion of the nucleotide G at position 732/733 in intron 2 of the hsgk1 gene.

25. A method for diagnosing hypertension in vitro, comprising the following procedural steps:
(a) obtaining a biological sample;
(b) isolating and/or amplifying genomic DNA, cDNA or mRNA from the biological sample obtained in (a); and
(c) quantifying in the biological sample the alleles which possess an insertion of the nucleotide G at position 732/733 in intron 2 of the hsgk1 gene.

26. The method as claimed in claim 24, wherein the biological sample from step (a) is selected from the group consisting of blood, saliva, tissue and cells.

27. The method as claimed in claim 25, wherein the biological sample from step (a) is selected from the group consisting of blood, saliva, tissue and cells.

28. The method as claimed in claim 24, wherein the alleles are quantified according to step (b) by directly sequencing the genomic DNA or cDNA which has been isolated from the biological sample.

29. The method as claimed in claim 25, wherein the alleles are quantified according to step (c) by directly sequencing the genomic DNA or cDNA which has been isolated from the biological sample.

30. The method as claimed in claim 24, wherein the alleles are quantified according to step (b) by specifically hybridizing the genomic DNA or cDNA which has been isolated from the biological sample.

31. The method as claimed in claim 25, wherein the alleles are quantified according to step (c) by specifically hybridizing the genomic DNA or cDNA which has been isolated from the biological sample.

32. The method as claimed in claim 24, wherein the alleles are quantified according to step (b) by means of a PCR oligo elongation assay or a ligation assay.

33. The method as claimed in claim 25, wherein the alleles are quantified according to step (c) by means of a PCR oligo elongation assay or a ligation assay.

34. A method of using the direct correlation between the overexpression or functional molecular modification of human homologues of the sgk family and the length of the Q/T interval for diagnosing the long QT syndrome in vitro.

35. A method of using the single-stranded or double-stranded nucleic acid comprising the sequence of a human homologue of the sgk family or one of its fragments having a length of at least 10 nucleotides/base pairs for diagnosing the long QT syndrome in vitro.

36. The method as claimed in claim 24, wherein the human homologue of the sgk family is the hsgk1 gene.

37. The method as claimed in claim 35, wherein the human homologue of the sgk family is the hsgk1 gene.

38. The method as claimed in claim 36, wherein the nucleic acid, the hsgk1 gene, or one of its fragments:
(a) possesses a length of at least 10 nucleotides/base pairs; and
(b) wherein said nucleic acid comprises the polymorphism at position 732/733 in intron 2 of the hsgk1 gene either with or without the insertion of the nucleotide G.

39. The method as claimed in claim 37, wherein the nucleic acid, the hsgk1 gene, or one of its fragments:
(a) possesses a length of at least 10 nucleotides/base pairs; and
(b) wherein said nucleic acid comprises the polymorphism at position 732/733 in intron 2 of the hsgk1 gene either with or without the insertion of the nucleotide G.

40. A method of using an antibody directed against Nedd 4-2 having the Acc. No. BAA23711 for diagnosing in vitro a predisposition for developing the long Q/T syndrome, with the antibody being directed against an epitope of the human homologue which contains the phosphorylation site either in phosphorylated form or in unphosphorylated form.

41. The kit for diagnosing the long QT syndrome, comprising:
(a) antibodies directed against the human homologues of the sgk protein family; or
(b) single-stranded or double-stranded nucleic acid fragments which: (i) are at least 10 nucleotides/base pairs in length and (ii) are able to hybridize, under stringent conditions, with the human homologues of the sgk gene family; or
(c) both (a) and (b).

42. The kit as claimed in claim 41, wherein the human homologue of the sgk family is the hsgk1 gene.

43. The kit as claimed in claim 42, comprising nucleic acid fragments as specific hybridization probes, which comprise at least one of the SNPs in the hsgk1 gene in exon 8 (C2617T, D240D), in intron 6 (T2071C) or in intron 2 at position 732/733 (6 insertion).

44. A method of using a functional activator, or a positive transcription regulator, of a human homologue of the sgk family for lowering the Q/T interval.

45. The method as claimed in claim 44, wherein the functional activator or positive transcription regulator is selected from the group consisting of glucocorticoids, mineralocorticoids, aldosterone, gonadotropins and cytokines.

46. A method of using substances selected from the group consisting of glucocorticoids, mineralocorticoids, aldosterone, gonadotropins and cytokines for producing a pharmaceutical for the therapy and/or prophylaxis of the long QT syndrome.

47. A pharmaceutical comprising at least one substance from the group of substances consisting of mineralocorticoids, aldosterone, gonadotropins and cytokines for the therapy, prophylaxis or therapy and prophylaxis of the long QT syndrome.

48. A pharmaceutical as claimed in claim 47, wherein the substance is TGF-β.

49. The method as claimed in claim 44, wherein the family is hsgk1.

50. The method as claimed in claim 45, wherein the activator or regulator is TGF-β.

51. The method as claimed in claim 46, wherein the substance is TGF-β.


SEQUENCE LISTING (2 sequences)

SEQ ID NO: 1 - DNA, 5704 bp, Homo sapiens
ggccgagcgcgcggcctggcgcacgatacgccgagccggtctttgagcgctaacgtctttctgtctccccgcggtggtgatgacggtgaaaactgaggctgctaagggcaccctcacttactccaggatgaggggcatggtggcaattctcatcggtgagtgcaggaatcttgcgggacttctgctccaggagacgcaaagtggaaattttttgaaagtcccggatcagattagtgtgtgtggcgccgggacgttatgaagccgtctaaacgtttctttatttctcctccttctatccacagctttcatgaagcagaggaggatgggtctgaacgactttattcagaagattgccaataactcctatgcatgcaaacagtaagttcagaccggattgaggaaataactagtatagtttgaatttgccagcggtaaacattctcatcacggcgtttatcgggaaggcgaagacttcttctggggtggggatctcatttctccttaaattctaatatatttgacacattttaaacattaaagttaatttgctgatttggcttgaactggagatgtaagataaatggttcgtgttggccgaattcacgctttctccatgagcaacaatccttatttctgtatttaatggggtttattattttctttaactgactaatgtattggggtattttcagtttaaacagtgaattatcgggtagaagtcggtagagccagaaactcacttttgatgttggtgtgccccctagtggcgagctggattctaaatcgtgccctttattccctgcagccctgaagttcagtccatcttgaagatctcccaacctcaggagcctgagcttatgaatgccaacccttctcctccagtaagtttttgtatgtgccgtgcatctgtggagaactgtaagggagtcagttagtattcctacattaatggattaaaatagcatttctagaaattagtatcaaggcaggaatgcttcattatgcataacagtgatataaatatttaagtattgagtcagagtattatttttatttttttcctgggcatattttacctcaagtggttattttaaaaggcatatttcataaaaaggttttatctgtctgaaacaacatgactgtgtgcagtttccatactcatttgaaatgtgatgaaatgtagttttgaatgtttatagatgtatggtcatttgcatcagtcatttgtagatgtaacattttctacatcgtttatgttatagatgtcttcctttgaagcaatggtattaaaagaaattcctagccaagtccttctcagcaaatcaaccttggcccgtcgtccaatcctcatgctaaaccatctgactttcacttcttgaaagtgatcggaaagggcagttttggaaaggtaatttcaaatctgaagatcttttggtacacttccttcatgtcctcttttatattctccctggatgaggatcgaaaaatgatttttttaaattgaaatttcaggttcttctagcaagacacaaggcagaagaagtgttctatgcagtcaaagttttacagaagaaagcaatcctgaaaaagaaagaggtgagatgtgcttgatggggctggcattggcggtagacactccttgaataatcttgattctggaatgttggtgccagttgaacatgccactaaatctgaatcgtcattttcctaggagaagcatattatgtcggagcggaatgttctgttgaagaatgtgaagcaccctttcctggtgggccttcacttctctttccagactgctgacaaattgtactttgtcctagactacattaatggtggagaggtgagcaggggggatagaagtcaactcttagtgtctctgcacagcctgctttgttttagtttgagaaaaaagttttcaaagatttttggtggggagaatgttaccagaattagcatttccttcaacctgtcaggttatagttaatagattacttggggccacttcctgcagttgttcttttgctgtgtatgtcaaaactaattaaattacattgcgcaacccagaatgactttgttctgtctcctgcagttgttctaccatctccagagggaacgctgcttcctggaaccacgggctcgtttctatgctgctgaaatagccagtgccttgggctacctgcattcactgaacatcgtttataggtaagcctgagagctcttcaggctaccagttttggtataaaggagacgtagcactggctgtttcatagggccttaaaataatttgtgtttatttgcaacttggttcgctaaaaccagatcccctagcacgtgagctggcttgacttaagtgccaagggggaacagccaagtaggattgtgcctaatccagaatagatgagcagaacaagggctccttttttcttcactacacaactacagtgaacctaaatgcctctaataccttagcaattatctttaagaggatatcttatgaagtgaaattaacttgtgcaactacttttctttcacttttttacagagacttaaaaccagagaatattttgctagattcacagggacacattgtccttactgatttcggactctgcaaggagaacattgaacacaacagcacaacatccaccttctgtggcacgccggaggtaggcgctgtcttggtttggtgcctggtttacccccgccttccaagagagagatgtacaatcatgcacttaactaccaaaaagagtaaactcctctcagagacttcttaatacagttcagtgcaaataaaatacatttgctgtttgatgtagcatgagaaatcccaagtccttctgttcctttactgaaaagtagctgtttgtaagtaagatctgcatcataaaaactttctaatcctaagtaagagatatcaagtgccagcagtttcctaaatgtcagtacacataggtagccagtcaccctcaaaaagtccagcagttttatcaggaaggaatctaaagatatctatcttccaagctggctctgggtctctcagctttttcaaactaaatgtgtggtcgtgggattgcttgctttcgcaggttctaaacgctgtttccctggtctgtttttcagtatctcgcacctgaggtgcttcataagcagccttatgacaggactgtggactggtggtgcctgggagctgtcttgtatgagatgctgtatggcctggtgagtggcacattgggaaccactggaacactgcctgctccctacaatattgccttcacacagcaaaagcagctaagaggcatattggttattttatagttcataagaataatcacttacctggttcttttgtgcatttcacattttactagataggaccacattgaacctgtgtggtggtgaaaaactaccacttattaacatctaccccctaccctccacacacacacacacaaacacacacacgggttgcaaagtagacacttaaatagcaagggaaaagaaagcattgaggtggggagagtttctcaaatcgagcctaatatttattgccgtttatatctttttctctactggtaatgtgtgccatatgaaacttccaattaagtctaaagtaattttccccttctttcagccgcctttttatagccgaaacacagctgaaatgtacgacaacattctgaacaagcctctccagctgaaaccaaatattacaaattccgcaagacacctcctggagggcctcctgcagaaggacaggacaaagcggctcggggccaaggatgacttcgtgagtgatgttttcctgtcctcctgggccggccgggacgtgcactagacctccctgcccttattgaatgcacctgtctaaattaatcttgggtttcttatcaacagatggagattaagagtcatgtcttcttctccttaattaactgggatgatctcattaataagaagattactcccccttttaacccaaatgtggtgagtatctgtctctcttctaagtatagagaagccaagcgatttattttaattcagaattgtctgggggagggttggaaggaatacattggcagatgttttctccataaacctgttattttacctacatagacacatttatcaattcgaagcaccaaaaggcaacaagtgaacattattcttatgtttaactgtgtgtagccttttgagattttgtgcttgaagtgggtgattatggaagttgatataagacttaaacttggtatttaaagcctggtcaagatttccctgtcctgtgtctagtgtgagttcttgacaagagtgtttttcccttcccgtcacagagtgggcccaacgagctacggcactttgaccccgagtttaccgaagagcctgtccccaactccattggcaagtcccctgacagcgtcctcgtcacagccagcgtcaaggaagctgccgaggctttcctaggcttttcctatgcgcctcccacggactctttcctctgaaccctgttagggcttggttttaaaggattttatgtgtgtttccgaatgttttagttagccttttggtggagccgccagctgacaggacatcttacaagagaatttgcacatctctggaagcttagcaatcttattgcacactgttcgctggaattttttgaagagcacattctcctcagtgagctcatgaggttttcatttttattcttccttccaacgtggtgctatctctgaaacgagcgttagagtgccgccttagacggaggcaggagtttcgttagaaagcggacctgttctaaaaaaggtctcctgcagatctgtctgggctgtgatgacgaatattatgaaatgtgccttttctgaagagattgtgttagctccaaagcttttcctatcgcagtgtttcagttctttattttcccttgtggatatgctgtgtgaaccgtcgtgtgagtgtggtatgcctgatcacagatggattttgttataagcatcaatgtgacacttgcaggacactacaacgtgggacattgtttgtttcttccatatttggaagataaatttatgtgtagacttttttgtaagatacggttaataactaaaatttattgaaatggtcttgcaatgactcgtattcagatgcctaaagaaagcattgctgctacaaatatttctatttttagaaagggtttttatggaccaatgccccagttgtcagtcagagccgttggtgtttttcattgtttaaaatgtcacctgtaaaatgggcattatttatgtttttttttttgcattcctgataattgtatgtattgtataaagaacgtctgtacattgggttataacactagtatatttaaacttacaggcttatttgtaatgtaaaccaccattttaatgtactgtaattaacatggttataatacgtacaatccttccctcatcccatcacacaactttttttgtgtgtgataaactgattttggtttgcaataaaaccttgaaaaatatttacatatattgtgtcatgtgttattttgtatattttggttaagggggtaatcatgggttagtttaaaattgaaaaccatgaaaatcctgctgtaatttcctgcttagtggtttgctccaacagcagtggtttctgactccagggagtataggatggcttaagccaccacgtccaggcctttagcagcatt

SEQ ID NO: 2 - DNA, 5701 bp, Homo sapiens
ggccgagcgcgcggcctggcgcacgatacgccgagccggtctttgagcgctaacgtctttctgtctccccgcggtggtgatgacggtgaaaactgaggctgctaagggcaccctcacttactccaggatgaggggcatggtggcaattctcatcggtgagtgcaggaatcttgcgggacttctgctccaggagacgcaaagtggaaattttttgaaagtcccggatcagattagtgtgtgtggcgccgggacgttatgaagccgtctaaacgtttctttatttctcctccttctatccacagctttcatgaagcagaggaggatgggtctgaacgactttattcagaagattgccaataactcctatgcatgcaaacagtaagttcagaccggattgaggaaataactagtatagtttgaatttgccagcggtaaacattctcatcacggcgtttatcgggaaggcgaagacttcttctggggtggggatctcatttctccttaaattctaatatatttgacacattttaaacattaaagttaatttgctgatttggcttgaactggagatgtaagataaatggttcgtgttggccgaattcacgctttctccatgagcaacaatccttatttctgtatttaatggggtttattattttctttaactgactaatgtattggggtattttcagtttaaacagtgaattatcgggtagaagtcggtagagccaggaaactcacttttgatgttggtgtgccccctagtggcgagctggattctaaatcgtgccctttattccctgcagccctgaagttcagtccatcttgaagatctcccaacctcaggagcctgagcttatgaatgccaacccttctcctccagtaagtttttgtatgtgccgtgcatctgtggagaactgtaagggagtcagttagtattcctacattaatggattaaaatagcatttctagaaattagtatcaaggcaggaatgcttcattatgcataacagtgatataaatatttaagtattgagtcagagtattatttttatttttttcctgggcatattttacctcaagtggttattttaaaaggcatatttcataaaaaggttttatctgtctgaaacaacatgactgtgtgcagtttccatactcatttgaaatgtgatgaaatgtagttttgaatgtttatagatgtatggtcatttgcatcagtcatttgtagatgtaacattttctacatcgtttatgttatagatgtcttcctttgaagcaatggtattaaaagaaattcctagccaagtccttctcagcaaatcaaccttggcccgtcgtccaatcctcatgctaaaccatctgactttcacttcttgaaagtgatcggaaagggcagttttggaaaggtaatttcaaatctgaagatcttttggtacacttccttcatgtcctcttttatattctccctggatgaggatcgaaaaatgatttttttaaattgaaatttcaggttcttctagcaagacacaaggcagaagaagtgttctatgcagtcaaagttttacagaagaaagcaatcctgaaaaagaaagaggtgagatgtgcttgatggggctggcattggcggtagacactccttgaataatcttgattctggaatgttggtgccagttgaacatgccactaaatctgaatcgtcattttcctaggagaagcatattatgtcggagcggaatgttctgttgaagaatgtgaagcaccctttcctggtgggccttcacttctctttccagactgctgacaaattgtactttgtcctagactacattaatggtggagaggtgagcaggggggatagaagtcaactcttagtgtctctgcacagcctgctttgttttagtttgagaaaaaagttttcaaagatttttggtggggagaatgttaccagaattagcatttccttcaacctgtcaggttatagttaatagattacttggggccacttcctgcagttgttcttttgctgtgtatgtcaaaactaattaaattacattgcgcaacccagaatgactttgttctgtctcctgcagttgttctaccatctccagagggaacgctgcttcctggaaccacgggctcgtttctatgctgctgaaatagccagtgccttgggctacctgcattcactgaacatcgtttataggtaagcctgagagctcttcaggctaccagttttggtataaaggagacgtagcactggctgtttcatagggccttaaaataatttgtgtttatttgcaacttggttcgctaaaaccagatcccctagcacgtgagctggcttgacttaagtgccaagggggaacagccaagtaggattgtgcctaatccagaatagatgagcagaacaagggctccttttttcttcactacacaactacagtgaacctaaatgcctctaataccttagcaattatctttaagaggatatcttatgaagtgaaattaacttgtgcaactacttttctttcacttttttacagagacttaaaaccagagaatattttgctagattcacagggacacattgtccttactgatttcggactctgcaaggagaacattgaacacaacagcacaacatccaccttctgtggcacgccggaggtaggcgctgtcttggtttggtgcctggtttacccccgccttccaagagagagatgtacaatcatgcacttaactaccaaaaagagtaaactcctctcagagacttcttaatacagttcagtgcaaataaaatacatttgctgtttgatgtagcatgagaaatcccaagtccttctgttcctttactgaaaagtagctgtttgtaagtaagatctgcatcataaaaactttctaatcctaagtaagagatatcaagtgccagcagtttcctaaatgtcagtacacataggtagccagtcaccctcaaaaagtccagcagttttatcaggaaggaatctaaagatatctatcttccaagctggctctgggtctctcagctttttcaaactaaatgtgtggtcgtgggattgcttgctttcgcaggttctaaacgctgtttccctggtctgtttttcagtatctcgcacctgaggtgcttcataagcagccttatgacaggactgtggactggtggtgcctgggagctgtcttgtatgagatgctgtatggcctggtgagtggcacattgggaaccactggaacactgcctgctccctacaatattgccttcacacagcaaaagcagctaagaggcatattggttattttatagttcataagaataatcacttacctggttcttttgtgcatttcacattttactagataggaccacattgaacctgtgtggtggtgaaaaactaccacttattaacatctaccccctaccctccacacacacacacacaaacacacacacgggttgcaaagtagacacttaaatagcaagggaaaagaaagcattgaggtggggagagtttctcaaatcgagcctaatatttattgccgtttatatctttttctctactggtaatgtgtgccatatgaaacttccaattaagtctaaagtaattttccccttctttcagccgcctttttatagccgaaacacagctgaaatgtacgacaacattctgaacaagcctctccagctgaaaccaaatattacaaattccgcaagacacctcctggagggcctcctgcagaaggacaggacaaagcggctcggggccaaggatgacttcgtgagtgatgttttcctgtcctcctgggccggccgggacgtgcactagacctccctgcccttattgaatgcacctgtctaaattaatcttgggtttcttatcaacagatggagattaagagtcatgtcttcttctccttaattaactgggatgatctcattaataagaagattactcccccttttaacccaaatgtggtgagtatctgtctctcttctaagtatagagaagccaagcgatttattttaattcagaattgtctgggggagggttggaaggaatacattggcagatgttttctccataaacctgttattttacctacatagacacatttatcaattcgaagcaccaaaaggcaacaagtgaacattattcttatgtttaactgtgtgtagccttttgagattttgtgcttgaagtgggtgattatggaagttgatataagacttaaacttggtatttaaagcctggtcaagatttccctgtcctgtgtctagtgtgagttcttgacaagagtgtttttcccttcccgtcacagagtgggcccaacgagctacggcactttgaccccgagtttaccgaagagcctgtccccaactccattggcaagtcccctgacagcgtcctcgtcacagccagcgtcaaggaagctgccgaggctttcctaggcttttcctatgcgcctcccacggactctttcctctgaaccctgttagggcttggttttaaaggattttatgtgtgtttccgaatgttttagttagccttttggtggagccgccagctgacaggacatcttacaagagaatttgcacatctctggaagcttagcaatcttattgcacactgttcgctggaattttttgaagagcacattctcctcagtgagctcatgaggttttcatttttattcttccttccaacgtggtgctatctctgaaacgagcgttagagtgccgccttagacggaggcaggagtttcgttagaaagcggacctgttctaaaaaaggtctcctgcagatctgtctgggctgtgatgacgaatattatgaaatgtgccttttctgaagagattgtgttagctccaaagcttttcctatcgcagtgtttcagttctttattttcccttgtggatatgctgtgtgaaccgtcgtgtgagtgtggtatgcctgatcacagatggattttgttataagcatcaatgtgacacttgcaggacactacaacgtgggacattgtttgtttcttccatatttggaagataaatttatgtgtagacttttttgtaagatacggttaataactaaaatttattgaaatggtcttgcaatgactcgtattcagatgcctaaagaaagcattgctgctacaaatatttctatttttagaaagggtttttatggaccaatgccccagttgtcagtcagagccgttggtgtttttcattgtttaaaatgtcacctgtaaaatgggcattatttatgtttttttttttgcattcctgataattgtatgtattgtataaagaacgtctgtacattgggttataacactagtatatttaaacttacaggcttatttgtaatgtaaaccaccattttaatgtactgtaattaacatggttataatacgtacaatccttccctcatcccatcacacaactttttttgtgtgtgataaactgattttggtttgcaataaaaccttgaaaaatatttacatatattgtgtcatgtgttattttgtatattttggttaagggggtaatcatgggttagtttaaaattgaaaaccatgaaaatcctgctgtaatttcctgcttagtggtttgctccaacagcagtggtttctgactccagggagtataggatggcttaagccaccacgtccaggcctttagcag